(19) United States
(12) Patent Application Publication
(10) Pub. No.: US 2011/0033894 A1
(43) Pub. Date: Feb. 10, 2011

(54) ENGINEERING SURFACE EPITOPES TO IMPROVE PROTEIN CRYSTALLIZATION

(76) Inventors: William N. Price, II, Barnesville, MD (US); John F. Hunt, III, New York, NY (US)

Correspondence Address: WilmerHale/Columbia University, 399 PARK AVENUE, NEW YORK, NY, 10022, US

(21) Appl. No.: 12/759,571

(22) Filed: Apr. 13, 2010

Related U.S. Application Data
(60) Provisional application No. 61/168,819, filed on Apr. 13, 2009.

(51) Int. Cl. C12P 21/02; C07K 14/00; B01J 19/00

(52) U.S. Cl. 435/71.1; 530/402; 422/129

(57) ABSTRACT

Methods and systems for engineering surface epitopes, based on protein sequence characteristics with statistically significant influence on the likelihood of x-ray structure solution, to improve protein crystallization, as well as related material, are disclosed.

### REFERENCE TO RELATED APPLICATION

This application claims benefit of priority to U.S. Provisional Patent Application No. 61/168,819, filed Apr. 13, 2009, the entire disclosure of which is hereby incorporated by reference.

### GOVERNMENT INTERESTS

The work described herein was supported in whole, or in part, by National Institute of Health Grant Nos. GM074958 and GM072867. Thus, the United States Government has certain rights to the invention.

### BACKGROUND

Current understanding of biology makes great use of atomic level protein structures, but the generation of these structures, e.g., by x-ray crystallography, is both expensive and uncertain. A significant bottleneck in the process is the generation of high quality crystals for x-ray diffraction. Much effort has gone to developing crystallization screens, and to creating high-throughput methods for cloning and expressing proteins (see, e.g., Acton T. B. et al., Methods Enzymol. 2005, 394, 210-243). However, the mechanisms of crystallization—and the protein characteristics that impact it—remain largely unknown and poorly understood, with different methods of study yielding substantially different results.

### BRIEF DESCRIPTION OF THE FIGURES

FIG. 1 is a graphic representation showing sequence characteristics and correlation with crystal structure solution. For 679 proteins screened for crystallization, logistic regressions were calculated between sequence characteristics and crystal structure solution. Variables considered included the fractional content of each amino acid, mean residue hydrophobicity (GRAVY), length, mean residue charge, pI, net charge, and mean side chain entropy (SCE). Characteristics are ordered by: FIG. 1a, predictive value, measured as the standard deviation of the variable multiplied by the slope of its logistic regression; FIG. 1b, slope of the logistic regression; FIG. 1c, negative log of the p value for the logistic regression; p=0.01 is indicated by the dotted line.

FIG. 2 is a graphic representation showing four major predictors of crystal structure solution. Boxes indicate the fraction of proteins with PDB structures, binned by the predictor variable, and whiskers show 95% confidence intervals. Lines trace the logistic regression for each variable. FIG. 2a: GRAVY positively predicts, p=0.0000135. FIG. 2b: mean SCE of Prof/PhD predicted exposed residues negatively predicts, p=0.0001. FIG. 2c: fractional glycine among Prof/PhD predicted buried residues positively predicts, p=0.0005. FIG. 2d: fraction of residues predicted disordered by DISOPRED negatively predicts, p=0.0003.

FIG. 3 is a graphic representation showing combined predictive metric performance and validation. Boxes and whiskers show fraction in PDB and 95% confidence intervals in black for the 679 development proteins, and in grey the fraction for the 200 subsequent validation proteins, binned by Pxs, the predicted fraction of proteins yielding a solved crystal structure. The upper line shows a perfect predictor (pmodel=0.0000000053), and the lower line the trend of the validation data (p=0.0027).

FIG. 4 is a graphic representation showing protein stability and correlation with crystal structure solution. Fraction in PDB and 95% confidence intervals for stability bins are shown: FIG. 4a, melting temperature (p=0.0077, N=118; without hyperthermostable proteins, p=0.083, N=111); and FIG. 4b: ΔG of unfolding as measured by CD observation of guanidine denaturation (p=0.2, N=36).

FIG. 5 is a graphic representation showing solution properties and correlation with crystal structure solution. Bars indicate fraction in PDB, and whiskers show 95% confidence intervals. FIG. 5a: fraction in PDB by species in solution. Monomers produce PDB structures less readily than dimers (p=0.0005), than multimers (p=0.0002), and than the combination (p=0.000007). Dimers and multimers are not statistically significantly different (p=0.5). FIG. 5b: fraction in PDB by hydrodynamic homogeneity. Monodisperse proteins are significantly higher than all other solution states (p=0.01), and monodisperse and primary monodisperse proteins are higher than polydisperse and aggregated proteins (p=0.03). No other differences are statistically significant.

FIG. 6 is a graphic representation showing comparison of whole and fractional predictors. Negative log p values are shown for logistic regressions of whole and fractional predictors against crystal structure solution. For every significant predictor, fractional values are more significant than whole values.

FIG. 7 is a graphic representation showing significantly predictive individual amino acids. Boxes indicate the fraction of proteins with PDB structures, binned by fractional content of each amino acid; whiskers show 95% confidence intervals. Lines trace the logistic relationships for FIG. 7a: Ala (p=0.0019); FIG. 7b: Phe (p=0.015); FIG. 7c: Gly (p=0.0046); FIG. 7d: Glu (p=0.0029); and FIG. 7e: Lys (p=0.0018).

FIG. 8 is a graphic representation showing isoelectric point and correlation with crystal structure solution. The relationship between isolectric point and crystal structure solution is shown. FIG. 8a: a histogram of the 679 test proteins with and without PDB structures; FIG. 8b: a box and whiskers plot showing fraction in PDB and 95% confidence intervals for pI bins.

FIG. 9 is a graphic representation showing protein length and correlation with crystal structure solution. Distributions of protein length: FIG. 9a: the InPDB and OutPDB sets; FIG. 9b: the human and E. coli predicted soluble proteome and non-redundant PDB. Also shown are bins by protein length showing fraction in PDB and 95% confidence intervals: FIG. 9c: the test set; and FIG. 9d: the human and E. coli predicted soluble proteomes and non-redundant PDB. For the test set, the relationship appears bimodal, with an inflection point around 400 amino acids. The black line in FIG. 9c traces a regression of proteins under 400 a.a. in length (p=0.024), and the grey that of proteins over 400 a.a. (p=0.11). Length has no significant predictive effect for the E. coli proteome; for the human proteome the regression line is highly significant (p=7.8×10⁻⁵²).

FIG. 10 is a graphic representation showing charge variables and correlation with crystal structure solution. Box and whisker plots show fraction of proteins with PDB structures and 95% confidence intervals for whole and fractional values of charged residues (FIG. 10a, FIG. 10b), net charge (FIG. 10c, FIG. 10d), absolute net charge (FIG. 10e, FIG. 10f), positive residues (FIG. 10g, FIG. 10h), and negative residues (FIG. 10i, FIG. 10j).

FIG. 11 is a graphic representation showing absence of correlation between hydrophobicity and stability. FIG. 11a: the scatterplot of GRAVY vs. melting temperature, as measured by SYPO Orange dye-labeled thermal denaturation, shows no significant correlation (Pearson=0.12, N=118, p=0.19). FIG. 11b: the scatterplot of GRAVY vs. ΔG (unfolding), measured by CD observations of GuHCl denaturation, shows no significant correlation (Pearson=−0.059, N=36, p=0.73).

FIG. 12 is a graphic representation showing folded/unfolded segregation by charge and hydrophobicity. Unfolded proteins (open circles), folded proteins (filled circles), and aggregated proteins (triangles) are plotted by absolute mean net charge and mean normalized hydrophobicity. The black line has been posited by Uversky to divide charge/hydrophobicity space into an unfolded region below the line and a folded region above the line. A Fisher's exact test of the proteins shown here did not support that hypothesis (p=1).

FIG. 13 is a graphic representation showing functional form of the combined predictive metric. FIG. 13a: box and whiskers plot of fraction in PDB with 95% confidence intervals for Pxs bins, with the line tracing the multiple logistic regression (p=0.0000000053); FIG. 13b: functional form of Pxs, where the variables are mean side chain entropy of Prof/PhD predicted exposed residues, percent glycine among predicted buried residues, percent residues predicted disordered by DISOPRED, and percent phenylalanine.

FIG. 14 is a graphic representation showing application of Pₓₛ to the E. coli and human proteomes. FIG. 14a: box and whisker plots show fraction in PDB, binned by Pxs, for the E. coli and human predicted soluble proteomes. Logistic regression lines are shown for E. coli (p=0.07) and humans (p=6.7×10⁻³⁶). FIG. 14b and FIG. 14c: fraction in PDB is graphed as a function of the rolling average of 2000-protein bins for the human and E. coli predicted soluble proteomes; the same logistic regression lines are shown as in FIG. 14a.

FIG. 15 is a graphic representation showing complex variable proteomic distributions. Distributions are shown for several variables for the human and E. coli predicted soluble proteomes and redundancy-culled X-ray PDB structures, the human predicted soluble proteome and redundancy-culled X-ray PDB structures with all sequences removed which were predicted to be above 25% disordered, and the original 679 development proteins. Distributions are shown for FIG. 15a: mean residue hydrophobicity (GRAVY); FIG. 15b: mean SCE of Prof/PhD predicted exposed residues; FIG. 15c: fractional glycine content of Prof/PhD predicted buried residues; and FIG. 15d: fraction residues predicted to be disordered by DISOPRED.

FIG. 16 is a graphic representation showing amino acid content distributions. Distributions of the predicted soluble proteome and redundancy-culled X-ray PDB structures for human and E. coli. Distributions are shown for fractional content of FIG. 16a: alanine; FIG. 16b: phenylalanine; FIG. 16c: glutamic acid; FIG. 16d: glycine; and FIG. 16e: lysine.

FIG. 17 is a graphic representation showing enrichment of residues in disordered sequences. For the human and E. coli predicted soluble proteomes, the graph shows the ratio, for each amino acid, between the amino acid frequency in stretches of 10 or more residues predicted to be disordered and all other sequences. Glycine and proline are particularly enriched in human disordered sequences; glutamine, lysine, and methionine are less represented.

FIG. 18 is a graphic representation showing a human predictive metric. The predicted soluble human proteome and redundancy-culled human PDB were randomly divided into two equal sets, Development and Validation. Multiple logistic regressions were run on the Development set. The metric was then run on the Validation set. In FIG. 18a, fraction in PDB for PxsH bins is shown with 95% confidence intervals. The perfect prediction line, dashed, matches the development set (p=6.92×10⁻⁷³) It predicts the Test set nearly as well (grey line, p=1.61×10⁻⁶⁷). The model, shown in FIG. 18b, predicts likelihood of crystallization based on percent proline, percent glycine, percent glycine among buried residues, length, and the products of percent disordered residues and percent phenylananine, glycine, and proline.

FIG. 19 is a graphic representation showing limited proteolysis impact on protein crystallization. Fractions in PDB of 114 proteins subjected to limited proteolysis by Proteinase K and Trypsin are shown. Average scores were calculated for: FIG. 19a: percent of intact protein remaining; FIG. 19b: percent of protein remaining (all bands summed); FIG. 19c: dominant fragment size as a percentage of the intact protein; and FIG. 19d: number of bands visible. Whiskers indicate 95% confidence intervals, and the line in FIG. 19c traces the only significant logistic relationship. Slopes and p values for the logistic regressions are provided in the table at FIG. 19e.

FIG. 20 is a graphic representation showing correlations between stability measurements: FIG. 20a shows Pearson coefficients and p values between sets of stability measurements; scatterplots are shown for FIG. 20b, the significant correlation between ΔG (unfolding) and Tm; and FIG. 20c: the significant correlation between fraction Lys+Arg vs. percent of intact protein remaining after trypsin digestion.

### SUMMARY

The invention is based, in part, on the surprising discovery that replacement of certain amino acids present in certain secondary structure elements (such as loops) with more desirable residues significantly improves crystallization properties of the engineered protein for purposes of x-ray crystallographic studies.

In one aspect the invention provides a method of producing an engineered protein for high-resolution x-ray crystallographic structure determination, the method comprising: (a) selecting a protein of interest; (b) aligning the protein of interest with a homolog of the protein of interest; (c) predicting the secondary structure of the protein of interest; (d) identifying a target amino acid in the protein of interest that: (i) is part of a secondary structure element in the predicted secondary structure of the protein of interest; and (ii) is aligned with a replacement amino acid in the homolog of the protein of interest; and (e) replacing the target amino acid in the protein of interest with the replacement amino acid to provide an engineered protein of interest.

In another aspect the invention provides a method of producing an engineered protein for high-resolution x-ray crystallographic structure determination, the method comprising: (a) selecting a protein of interest; (b) aligning the protein of interest with an ortholog of the protein of interest; (c) predicting the secondary structure of the protein of interest; (d) identifying a target amino acid in the protein of interest that: (i) is part of a secondary structure element in the predicted secondary structure of the protein of interest; and (ii) is aligned with a replacement amino acid in the ortholog of the protein of interest; and (e) replacing the target amino acid in the protein of interest with the replacement amino acid to provide an engineered protein of interest.

In some embodiments the method further comprises expressing the engineered protein of interest in a cell.

In some embodiments the method further comprises crystallizing the expressed engineered protein of interest.

In some embodiments the secondary structure element is a surface-exposed loop.

In some embodiments the loop is 5 to 15 amino acids in length.

In some embodiments the target amino acid is selected from the group consisting of: isoleucine, leucine, valine, glutamic acid, and lysine. In some embodiments the target amino acid is isoleucine, leucine, or valine. In some embodiments the replacement amino acid is selected from the group consisting of: glycine, alanine, and phenylalanine. In some embodiments the replacement amino acid is glycine. In some embodiments the replacement amino acid is phenylalanine.

In some embodiments the method further comprises generating a nucleic acid sequence encoding the engineered protein of interest.

In another aspect, the invention provides a system for designing an engineered protein for high-resolution x-ray crystallographic structure determination, the system comprising a computer having a processor and computer-readable program code for performing any of the methods described above.

In yet another aspect, the invention provides a crystal of the engineered protein of interest produced by any of the methods described above.

### DETAILED DESCRIPTION

Much work has been done to understand the protein characteristics affecting crystallization, especially since the advent of structural genomics and its attendant data. For example, Canaves et al. identified biochemical characteristics correlated with protein crystallization, including hydropathy, isoelectric point, sequence length, and low-disorder regions (Canaves et al., J. Mol. Biol. 2004, 344, 977-991). The Center for Eukaryotic Structural genomics analyzed the effect of predicted disorder on crystallization (Oldfield, C. J. et al., Proteins 2005, 59, 444-453), and the previous work on the Northeast Structural Genomics (NESG) pipeline identified charged residues, binding partners, and sequence conservation as important predictive factors (Goh et al., J. Mol. Biol. 2004, 336, 115-130). Overton and Barton provided a scale based on hydrophobicity and isoelectric point (Overton, I. M. and Barton G. J., FEBS Lett. 2006, 580, 4005-4009). In addition, Slabinski et al. analyzed proteins in TargetDB, the structural genomics target database, using a binned probability method, and generated a combined “crystallization feasibility” measure based on several measures which showed significant differences in distribution across variation in a protein characteristic (Slabinski, L. L. et al., Protein Sci. 2007, 16, 2472-2482). These approaches have yielded much insight into the processes of protein production and crystallization, especially at a larger scale.

As described herein, focusing on a smaller scale, characteristics were evaluated that may have the most influence on the process of crystallization itself. Rather than working with a multi-consortium database, or across the entire production process, this evaluation was based only on proteins for which detailed data across the entire production and crystallization process was available. The NESG has taken over 2000 target proteins through a uniform production and crystallization pipeline (Acton T. B. et al., Methods Enzymol. 2005, 394, 210-243). These proteins have been rigorously analyzed throughout the process, so that protein status at each stage of the pipeline is recorded and available for data-mining analysis.

Logistic regression analysis was used on a sample of 679 proteins that went through the NESG pipeline. All 679 proteins were biochemically well-behaved—i.e., they expressed well, were highly soluble, and were predominantly monodisperse in solution. These proteins were subjected to a consistent set of crystallization screens. This process eliminated most of the external variables affecting protein crystallization, including solubility differences, expression levels, and variations in experimental procedure—the protein production level of variables—leaving those which impacted crystallization itself.

The 679 protein sequences were culled from the SPINE database and manually curated to verify predominant monodispersity, based on Wyatt-AKTA static light scattering traces. For consistency, tags were removed from the protein sequences.

Regression variables included amino acid frequency, as well as the bulk characteristics of hydrophobicity (GRAVY, the GRand AVerage of hydropathY), length, isoelectric point, net charge, fraction of residues predicted to be disordered by the program DISOPRED, and side chain entropy (SCE). Charge was calculated based solely on amino acid counts, considering only arginine, lysine, glutamic acid, and aspartic acid. Isoelectric point was calculated using the EMBOSS algorithm at ExPASy. GRAVY was calculated using the Kyte-Doolittle values of hydropathy (Kyte, J. and Doolittle, R. F. J. Mol. Biol. 1982, 157, 105-132). SCE values for the individual amino acids were taken from published values (Creamer, T. P. Proteins 2000, 40, 443-450). DISOPRED scores were calculated locally using the DISOPRED2 program with a 5% false positive rate (Ward, J. J., et al., Bioinformatics 2004, 20, 2138-2139). Calculations of predicted burial/exposure and secondary structure were performed with PhD/Prof (Rost, B. et al., Nucleic Acids Res. 2004, 32, W321-326). Exposed and buried fractions were calculated as fractions of total exposed or buried length; e.g., number of predicted buried alanines divided by the number of total predicted buried residues. For binned distribution graphs, bins were equally spaced and graphed by bin center; for terminal bins on unbounded variables, the bin center was calculated as the bin average.

Logistic regressions constrain the relationship into a relatively simple functional form, and consequently do not accurately capture multimodal effects. However, given the relatively small sample size and the low likelihood of accurately mapping local shifts in characteristic-space, logistic regression provides the most practical way of capturing the gross outline of the true underlying characteristic distributions. In the binary language of logistic regressions, proteins were considered successful if they yielded a crystal structure for deposition in the PDB. That is, target success was evaluated not on the formation of crystals, but rather on the formation of crystals of high enough quality to permit atomic resolution structure determination. Logistic regressions were performed in S-PLUS, and significance was determined by 2-tailed T-test. Ninety-five percent confidence intervals were calculated using the binomial distribution.

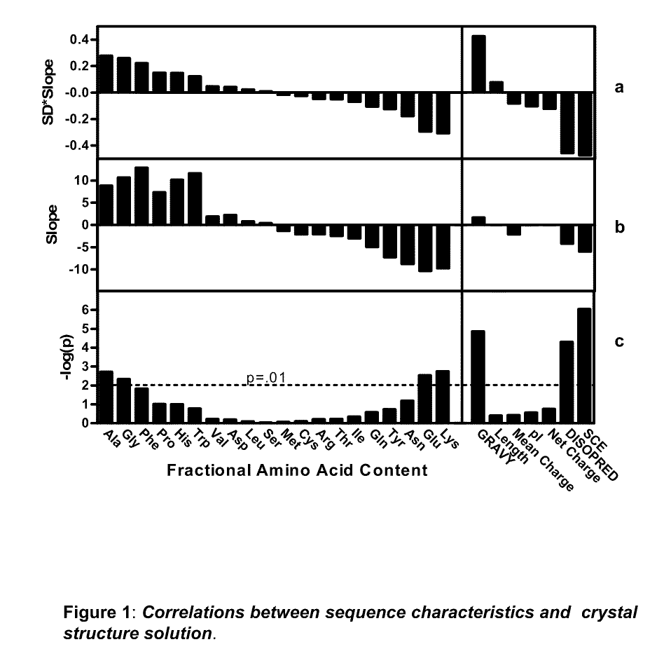

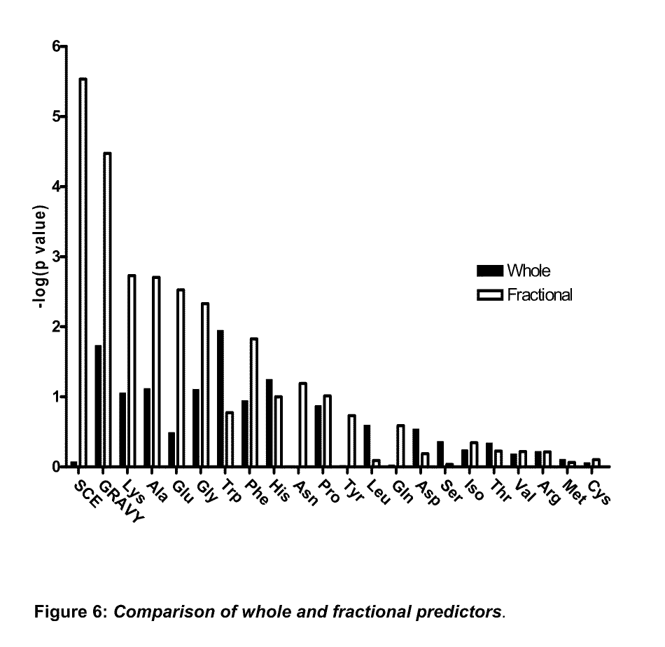

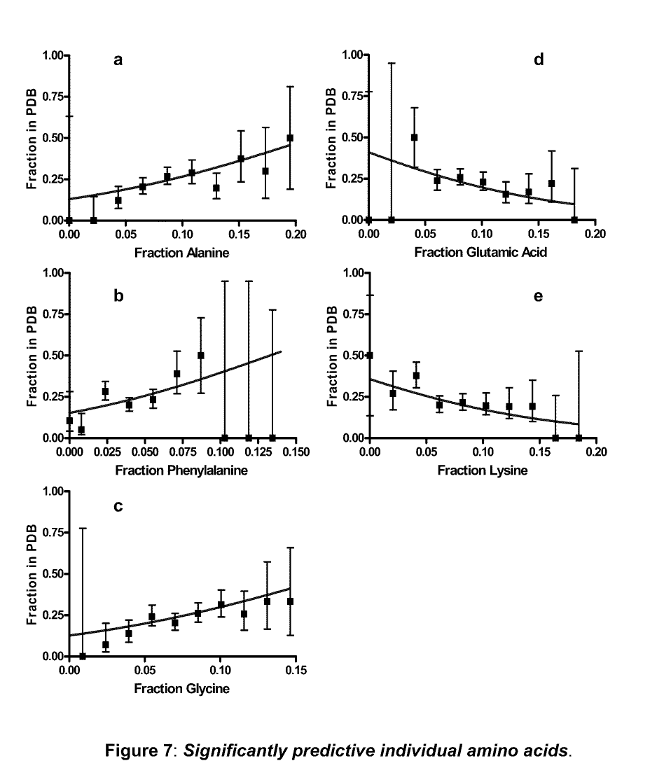

Predictive scores, regression slopes, and p values are presented in FIG. 1. Fractional values—i.e., mean residue SCE, fractional amino acid content—were more predictive than whole values, uniformly so for significant predictors (FIG. 6). Five amino acids were significantly predictive of crystal structure determination: glycine, phenylalanine, and alanine were positively correlated with structure determination, and glutamic acid and lysine were negatively correlated (FIG. 7). Table 1 shows parameters extracted from logistic regressions of 679 proteins against crystal structure solution.

TABLE 1
Table 1: Single and multiple logistic regressions.

| | Regression Variable | Slope | SD * Slope | P value |
|---|---|---|---|---|
| a | GRAVY | 1.68 | 0.43 | 0.0000135 |
| | SCE | −5.99 | −0.47 | 0.000000915 |
| | Gly | 10.685 | 0.26 | 0.0046 |
| | Pb SCE | −1.15 | −0.072 | 0.431 |
| | Pe SCE | −3.24 | −0.33 | 0.0001 |
| | Pb GRAVY | −0.413 | −0.25 | 0.0085 |
| | Pe GRAVY | 0.744 | 0.26 | 0.0044 |
| | Pb Gly | 9.16 | 0.32 | 0.0005 |
| | Pe Gly | 3.03 | 0.089 | 0.08 |
| b | DISOPRED | −4.21 | −0.46 | 0.0003 |
| | N/C/Internal DISOPRED | | | 0.0009 |
| | N-terminal DISOPRED | −4.37 | −0.18 | 0.15 |
| | C-terminal DISOPRED | −4.14 | −0.21 | 0.093 |
| | Internal DISOPRED | −4.19 | −0.31 | 0.013 |
| c | SCE + GRAVY | | | 0.00000181 |
| | SCE | −4.52 | −0.36 | 0.0066 |
| | GRAVY | 0.73 | 0.19 | 0.16 |
| d | Pb/e SCE + Amino Acids | | | 0.00192 |
| | Pb SCE | 2.52 | 0.16 | 0.22 |
| | Pe SCE | −4.18 | −0.43 | 0.017 |
| | Pb Ala | 2.61 | 0.12 | 0.30 |
| | Pe Ala | 1.14 | 0.044 | 0.71 |
| | Pb Glu | 5.05 | 0.049 | 0.64 |
| | Pe Glu | 0.0761 | 0.0030 | 0.98 |
| | Pb Gly | 11.2 | 0.39 | 0.00042 |
| | Pe Gly | −2.19 | −0.064 | 0.58 |
| | Pb Lys | −11.4 | −0.078 | 0.49 |
| | Pe Lys | 2.05 | 0.10 | 0.48 |

Logistic regressions for 679 proteins against crystal structure solution are shown.
Regressions are titled in bold; for multiple regressions, subvariables are listed separately.
Columns show the slope of the logistic regression; the predictive value, calculated as the product of the variable's standard deviation and regression slope, and the p value; p values below .05 are shown in boldface type.
a shows regressions for GRAVY, SCE, and fractional content of glycine, both whole and localized by Prof/PhD predicted burial (Pb) or exposure (Pe).
b shows regressions against fraction of residues predicted disordered by DISOPRED, both whole and divided by terminal or internal location.
c, a double regression with GRAVY and SCE, shows their redundant predictive signal.
d, a multiple regression of SCE and fractional glycine, segregated by Prof/PhD predicted burial/exposure, shows that predicted buried glycine and predicted exposed SCE have non-redundant predictive value.

Table 2 shows parameters extracted from logistic regressions of charge variables of 679 proteins against crystal structure solution.

TABLE 2
Table 2: Logistic Regressions of charge variables.

| Regression Variable | Slope | SD * Slope | P value |
|---|---|---|---|
| Number of Positive Residues | −0.00529 | −0.071 | 0.454 |
| Number of Negative Residues | −0.000367 | −0.0055 | 0.952 |
| Number of Charged Residues | −0.00133 | −0.037 | 0.692 |
| Net Charge | −0.0182 | −0.12 | 0.175 |
| Absolute Net Charge | 0.00270 | 0.014 | 0.876 |
| Fraction Positive Residues | −10.7 | −0.34 | 0.000665 |
| Fraction Negative Residues | −7.57 | −0.24 | 0.0144 |
| Fraction Charged Residues | −7.36 | −0.37 | 0.000249 |
| Fractional Net Charge | −2.13 | −0.081 | 0.376 |
| Fractional Absolute Net Charge | −1.47 | −0.042 | 0.650 |
| SCE & Frac. Pos./Neg. Residues | | | 0.0000192 |
| SCE | −5.58 | −0.441 | 0.0028 |
| Fraction Positive Residues | −1.37 | −0.044 | 0.742 |
| Fraction Negative Residues | −0.488 | −0.015 | 0.894 |

Slopes, predictive values, and p values for logistic regressions of various charge measurements against crystal structure solution are shown, with p values below .05 in boldface type.

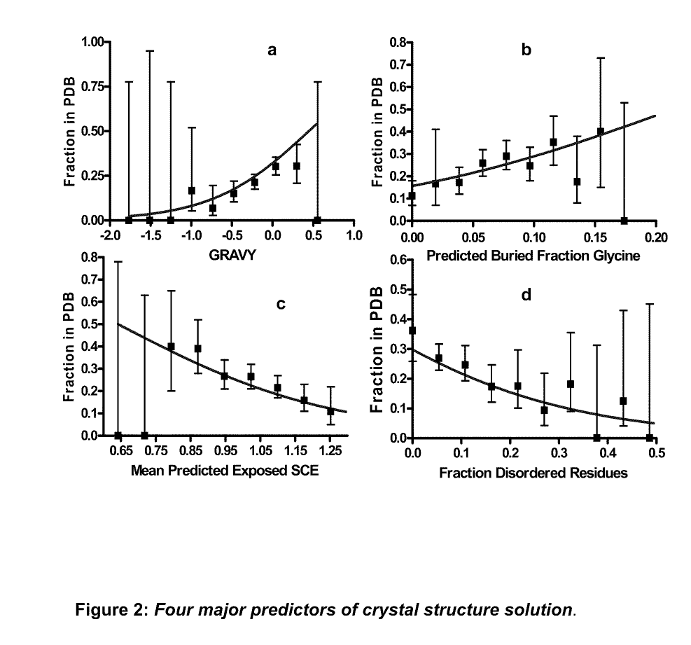

Three additional bulk characteristics were significantly correlated: GRAVY was positively correlated, predicted exposed SCE was negatively correlated, and the fraction of residues predicted to be disordered by the program DISOPRED were negatively correlated with structure determination (FIG. 2), whether those residues were terminal or internal (Table 1b).

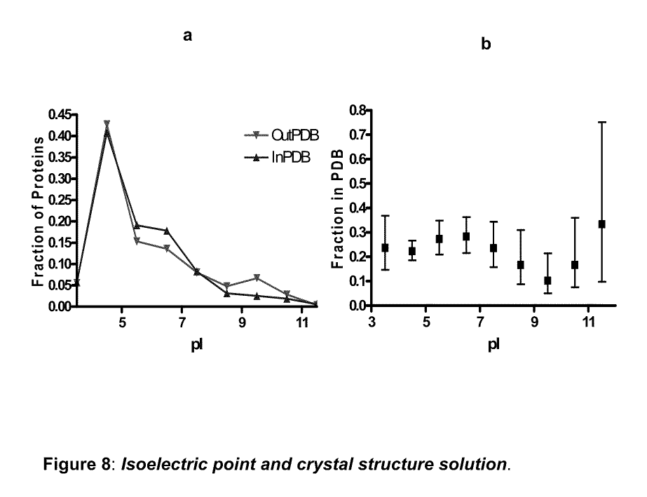

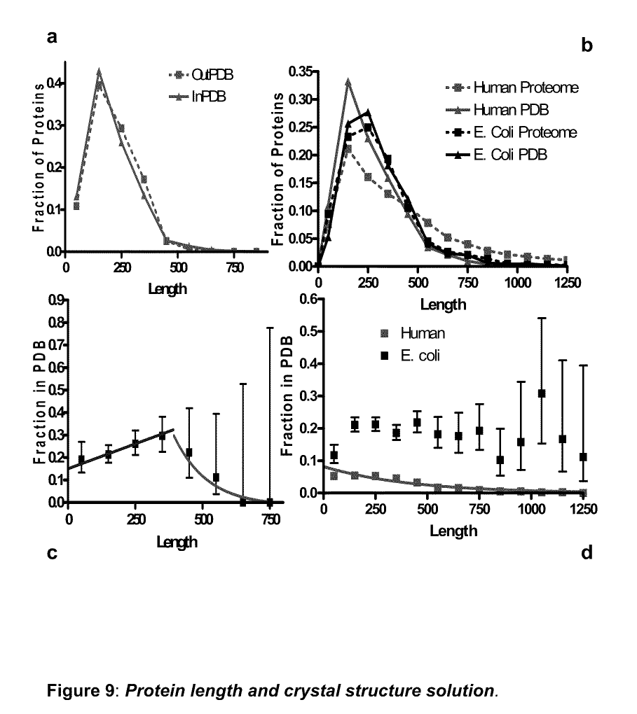

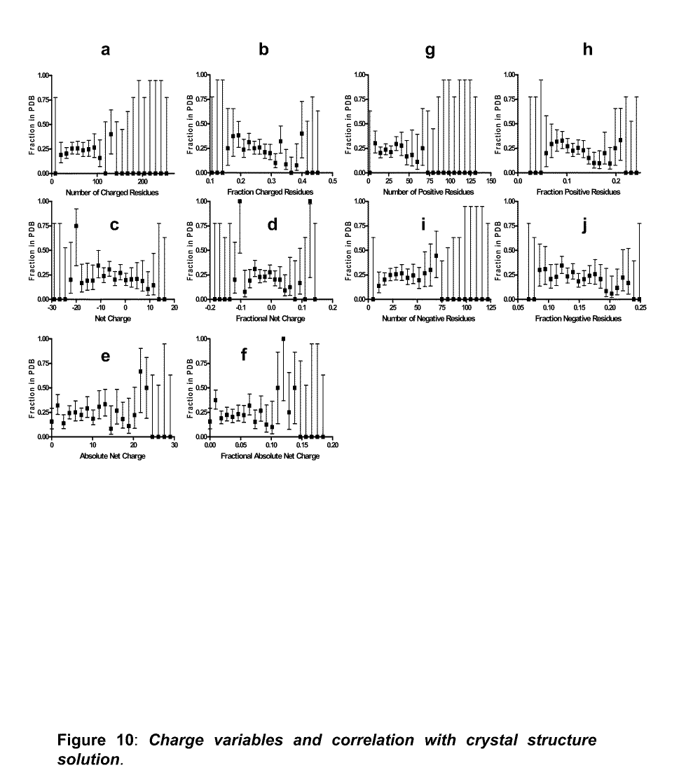

Isoelectric point (FIG. 8) and length (FIG. 9) both showed bimodal effects on crystal structure determination, but neither was highly significant, nor were they amenable to the logistic regression predictive methodology. Charge was not significantly predictive (Table 2) at a net level (FIG. 10a,c,e,g,i), nor were fractional net or absolute net charge (FIG. 10d,f). Fractional positive (FIG. 10h), negative (FIG. 10j), and total charged residues (FIG. 10b) were significantly negatively correlated, but this predictive information was redundant with that of SCE (FIG. 10k).

GRAVY and SCE were strongly predictive of crystal structure determination (Table 1a). Among the characteristics tested for affecting crystallization success, previous studies suggested that hydrophobicity had a strong impact on crystallization (JCSG OB-score; Canaves et al. 2004, J. Mol. Biol. 2004, 344, 977-991), and that the presence of amino acids with high side-chain entropy hinders crystallization (see, e.g., Derewenda, Z. S, and Vekilov P. G., Acta Cryst. 2006, D62, 116-124; Derewenda, Z. S., Structure 2004, 16, 529-535; Longenecker K. L. et al., Acta Cryst. 2001, D57, 679-688; surface entropy reduction prediction (SERp) server, Goldschmidt et al., Protein Science 2007, 16, 1569-1576). SCE and hydrophobicity are negatively correlated at the amino acid level and strongly negatively correlated at the protein level.

A double logistic regression showed that GRAVY and SCE had redundant predictive value, and thus may show different measures of the same mechanism by virtue of their high correlation (Table 1c). SCE effects on crystal packing may provide the dominant mechanism, with the observed effect of hydrophobicity being a correlation-based artifact. To identify the underlying mechanism, residues were segregated by burial or surface exposure as predicted by PhD/Prof.

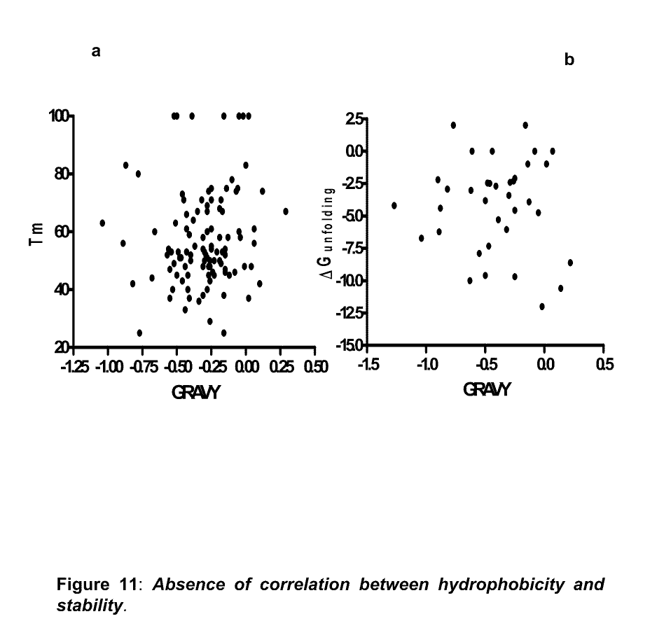

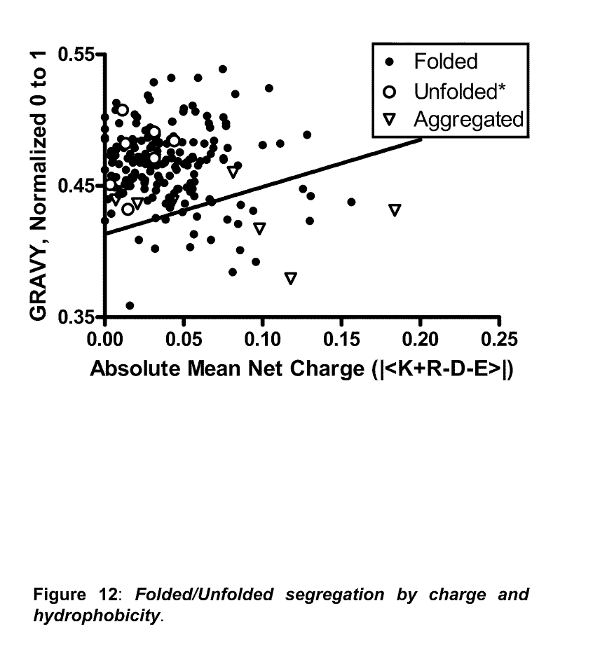

Hydrophobicity may influence high quality crystal formation through the presence of a more stable hydrophobic core, which may be evidenced by a correlation between observed stability and crystallizability, and by a localization of hydrophobicity's predictive signal to buried residues. GRAVY, however, predicted equally well when limited to either buried or exposed residues, and predicted exposed hydrophobicity favored crystallization, whereas predicted buried hydrophobicity opposed crystallization (Table 1a). Furthermore, GRAVY showed little correlation with thermal or chemical measures of stability (FIG. 11), and the combination of hydrophobicity and net charge (Uversky, V. N., Protein Sci. 2002, 11, 739-756) did not reliably separate our proteins into folded and unfolded (FIG. 12).

Side-chain entropy's influence, on the other hand, may center on the formation or prevention of high quality crystal packing contacts, and would therefore be localized to solvent-exposed residues. Mean side-chain entropy is only predictive in exposed residues (Table 1a). These data, taken together, imply that side-chain entropy, rather than hydrophobicity, strongly impacts the formation of high quality crystals, and that crystal packing contacts affect crystallization more than protein stability.

Some predictive signal may be redundant. Side chain entropy was negatively correlated with crystal structure determination, and four of the five significantly predictive amino acids have extreme SCE values: glycine and alanine, positively correlated, have zero SCE, while glutamic acid and lysine, negatively correlated, have high SCE. Multiple logistic regressions showed that the predictive signals from alanine, glutamic acid, lysine, and predicted exposed glycine were redundant with predicted exposed SCE, and that predicted buried glycine contact was a non-redundant and strongly positive signal (Table 1d). Since predictive signal of buried glycine is also largely localized to short loops between 5 and 15 residues, predicted buried glycine may be surface-exposed, and incorrectly predicted by the DSSP-based PhD/Prof model. Glycine may provide strong potential crystal packing sites, rather than serving to reduce a general entropic shield.

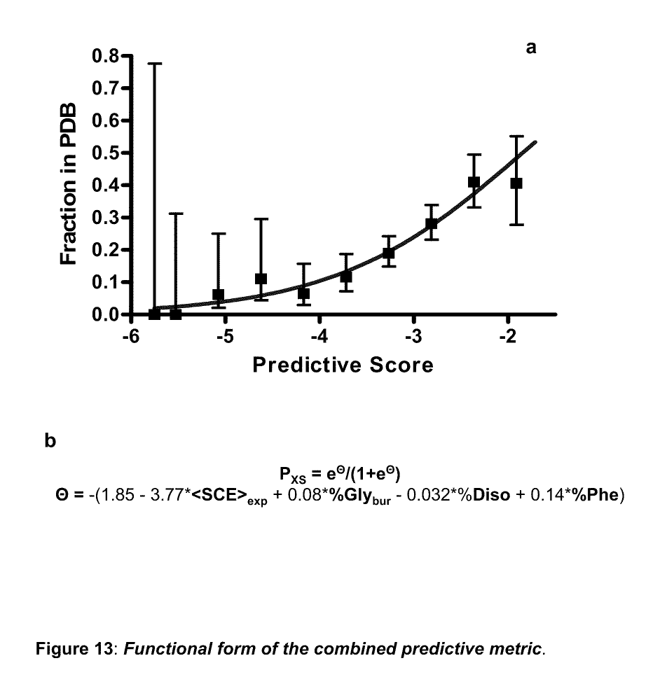

After eliminating redundancy among the significant predictive characteristics, a combined predictive metric was developed by performing a multiple logistic regression on the four non-redundant measures:

$${P}_{xs} = 1 / ( 1 + {e}^{- ( 1.8458 + 14.2629 * F - 3.2006 * D i so + 8.1380 * G - 3.7713 * SCE )} ) ,$$

where Pₓₛ is the probability of solving a crystal structure, F is fractional phenylalanine, Diso is fractional disordered residues, G is glycine as a fraction of buried residues, and SCE is mean predicted exposed side-chain entropy (FIG. 13).

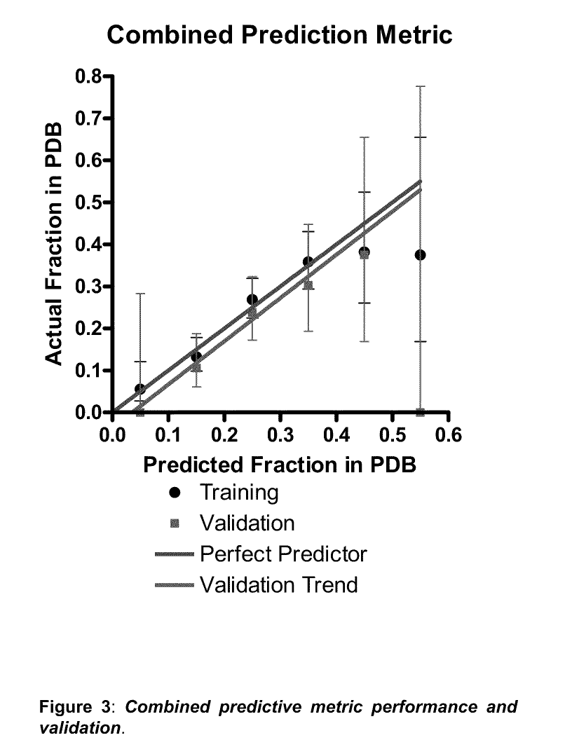

The Pₓₛ predictor provides an excellent description of the data (p=5.3×10⁻⁹, FIG. 3). The metric's predictive value was tested by calculating Pₓₛ for 200 proteins that passed through the NESG pipeline after the metric was developed (FIG. 3). Crystal structure determination increased linearly with increasing Pₓₛ (p=0.0014).

To further validate the metric, proteomic distributions of the predictive metric were calculated. Pₓₛ was determined for all predicted E. coli and human proteins without signal sequences or predicted transmembrane helices, and also for all human and E. coli PDB structures, culled for redundancy. Proteome data was taken from human and E. coli reference sequences. Sequences with one or more predicted transmembrane helices, as predicted by the program tmmhmm, were excluded from analysis. PDB sequences were downloaded Feb. 15, 2008, and are from the SEQRES database of x-ray structures. For proteome validation logistic regressions, all predicted protein sequences were assigned a value of zero, to avoid problems with construct variation and single-domain structure determinations. Rolling Pₓ, averages were calculated using bins of 2000 proteins for both humans and E. coli.

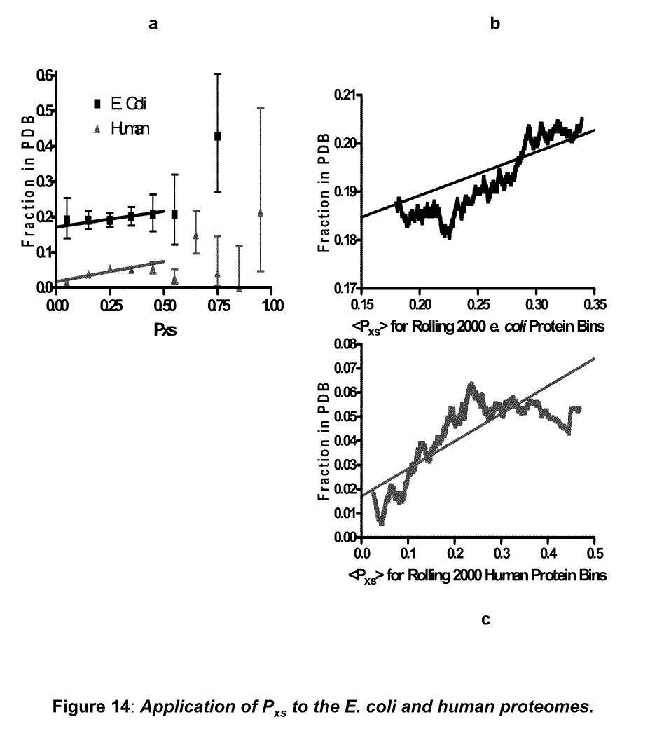

Pₓₛ was able to significantly predict which sequence characteristics were more likely to have PDB structures (FIG. 14), indicating that Pₓₛ, though developed from a uniformly treated and rigorously controlled set of proteins, has general applicability.

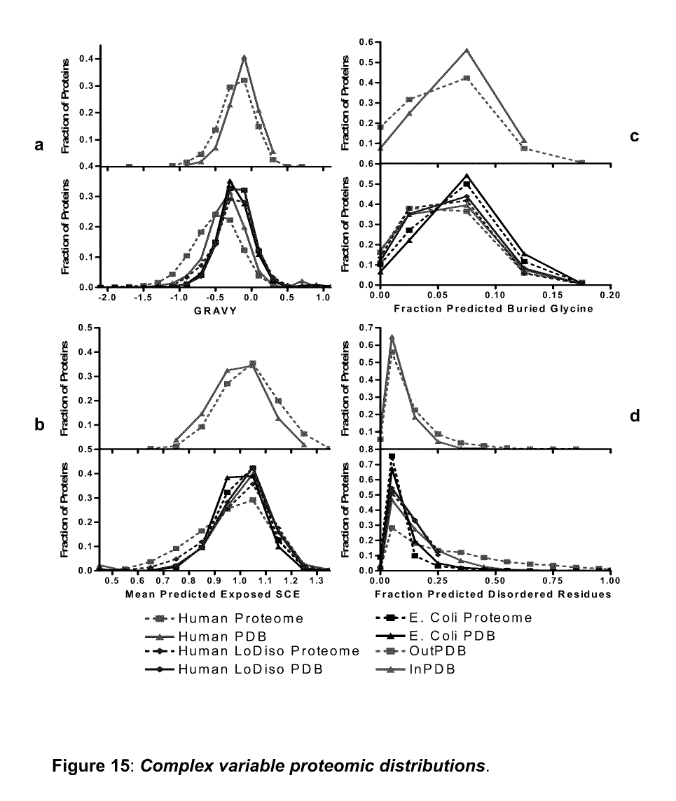

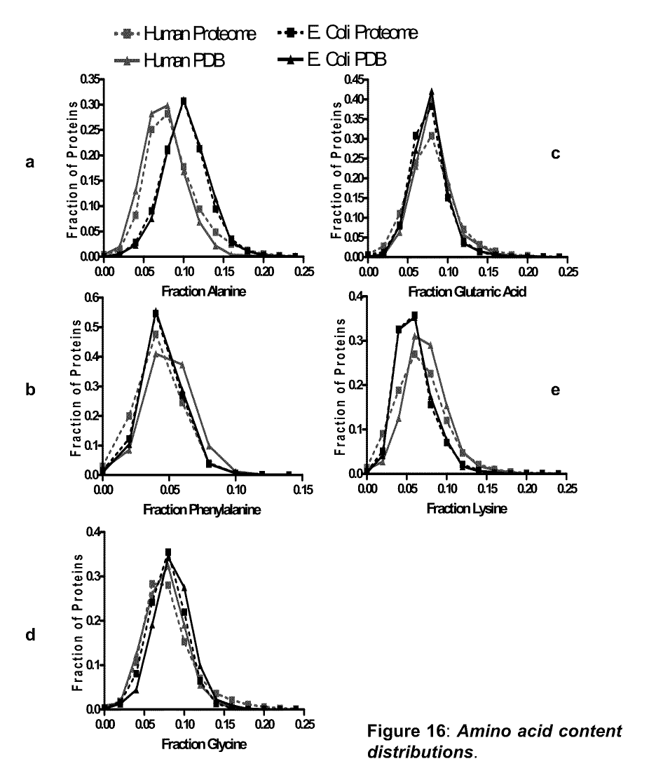

The sub-metrics of Pₓₛ were also evaluated, by calculating distributions for the four potential compound variables: GRAVY, predicted buried glycine, predicted exposed SCE, and predicted disordered residues (FIG. 15), and the five significant amino acids: alanine, phenylalanine, glutamic acid, glycine, and lysine (FIG. 16).

The E. coli distributions generally followed the trends seen in the primary dataset, though less dramatically; this effect may be because crystal structures have been solved for a high proportion of the E. coli genome proteins, leading to considerable sequence overlap in the two distributions.

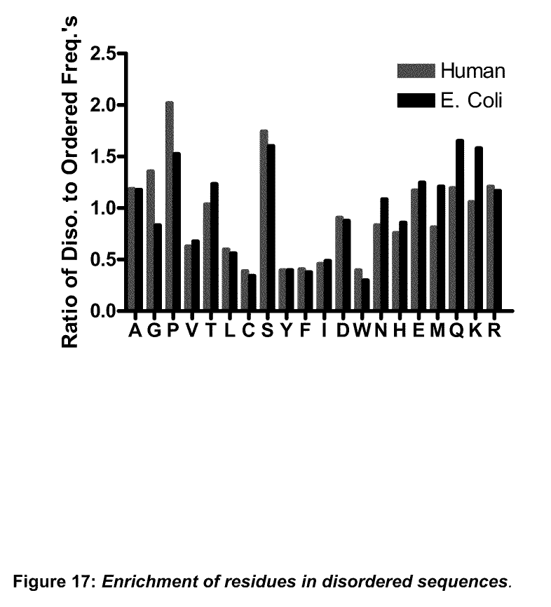

The human distributions followed the Pₓₛ trends in phenylalanine, buried glycine, and disordered residues, but human PDB structures had significantly fewer low SCE sequences than the human genome. Since solubility concerns were not included in the initial metric, there may be a greater advantage to higher-SCE charged residues in human proteins, which are often more difficult to express in high yields. In addition, human proteins have some fundamentally different sequence characteristics; they tend to have far more predicted disordered residues than bacterial proteins (FIG. 13d), and the exposed SCE effect largely disappears when those highly disordered sequences are excluded (FIG. 13c). Mechanistically, these disordered residues have a much higher prevalence of glycines and prolines (FIG. 17), two zero-SCE residues which, in high frequencies, seem mechanistically unlikely to aid crystallization, even though ordered glycines predicted to be “buried” in short loops are good for crystallization. Multiple logistic regressions confirmed that proline and glycine content in high disorder regions are particularly unfavorable for crystal structure determination.

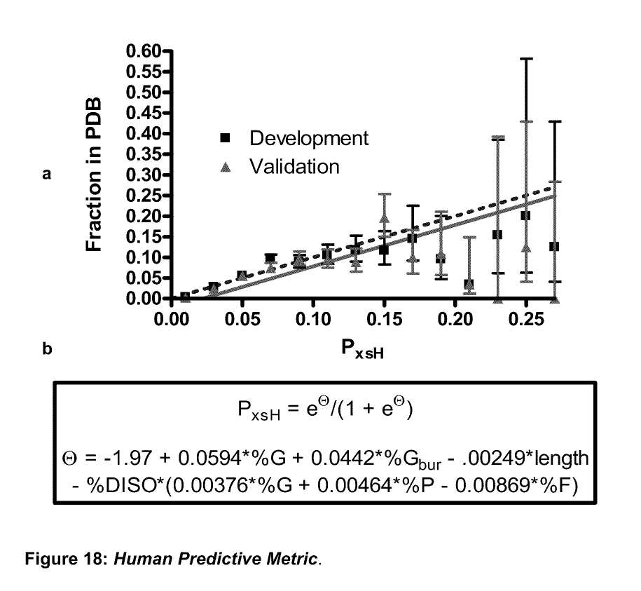

Since human proteins appear to behave differently from the bacterial proteins on which the primary analysis was largely based, another metric was developed for human proteins: Probability of Crystal Structure for Humans, P_{xsH}, based solely on the differences in proteomic and PDB sequences (FIG. 18). This metric is based on data from the entire solution procedure, not just crystallization, but may aid in selecting constructs for human protein crystallization.

Proteome and PDB data for P_{xsH} were collected as for Pₓₛ. Each sequence was randomly assigned to the Development set or the Validation set. Based on the enrichments of low-SCE residues shown in FIG. 17, multiple logistic regressions were run to analyze status as a PDB (1) or proteome (0) sequence based on fractions glycine, phenylalanine, and proline; fraction glycine among buried residues; fraction disordered residues; length; and the interaction terms of fraction disordered residues with the amino acid fractions. Insignificant variables were dropped from the regression and the regression was repeated to determine accurate coefficients. The combined metric was then used to predict and bin the Validation set.

The NESG pipeline has proven an extremely fruitful source of data on the process of protein crystallization itself. A metric has been developed that significantly predicts the formation of high quality crystals, and thus can be used to select targets for structural genomics or individual efforts at structure determination to avoid the prospect of failing at the final step of the pipeline. This model has rich mechanistic implications, which were supported by biophysical measurements described herein. Data described herein indicate that, rather than protein stability, the availability of residues for high quality crystal packing contacts is a dominant factor in the growth of crystals that diffract at high resolution. Residues with high side-chain entropy most likely impede the formation of these contacts, but glycine and phenylalanine appear to contribute positively beyond their low side-chain entropy, suggesting that they are themselves potential sites of crystal packing contacts. Proteins may be engineered based on these mechanistic implications to aid the future crystallization efforts.

The methods of the invention can be used to better understand the mechanism of crystallization, to achieve rational target selection in high-throughput drug screening endeavors, and to achieve mutants of targets in order to make them more easily druggable. In other words, the methods of this invention allow one to achieve an understanding of the active site and pocket structure, so that a drug which binds to that location (and possibly competes with a known ligand) can be identified. Simple metrics based on sequence characteristics can predict which proteins are more likely to yield crystal structures; these results are statistically rigorous and were validated in an independent set of proteins. Disordered residues and high side chain entropy residues negatively impact crystallization propensity; glycines in short loops and phenylalanine positively impact crystallization propensity. Oligomers crystallize more readily than monomers, and monodisperse proteins crystallize more readily than primarily monodisperse or polydisperse proteins. Protein stability, except for the extremes of hyperstable or partially unfolded proteins, appears to have little effect on crystallization propensity. These metrics can be used for target selection (sequence characteristics) and triage (biophysical characteristics), and will hopefully also yield novel target design strategies for challenging proteins.

Strategies and techniques for expressing a protein of interest, strategies and techniques for producing nucleic acids encoding a protein of interest are well-known in the art and can be found, e.g., in Berger and Kimmel, Guide to Molecular Cloning Techniques, Methods In Enzymology Vol. 152 Academic Press, Inc., San Diego, Calif. and in Sambrook et al. Molecular Cloning-A Laboratory Manual (2nd ed.) Vol. 1-3 (1989) and in Current Protocols In Molecular Biology, Ausubel, F. M., et al., eds., Greene Publishing Associates, Inc. and John Wiley & Sons, Inc., (1996 Supplement).

Equivalents: those skilled in the art will recognize, or be able to ascertain, using no more than routine experimentation, numerous equivalents to the specific embodiments described specifically herein. Such equivalents are intended to be encompassed in the scope of the following claims.

The disclosures of all patents, patent applications, and publications cited herein are hereby incorporated by reference in their entirety.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

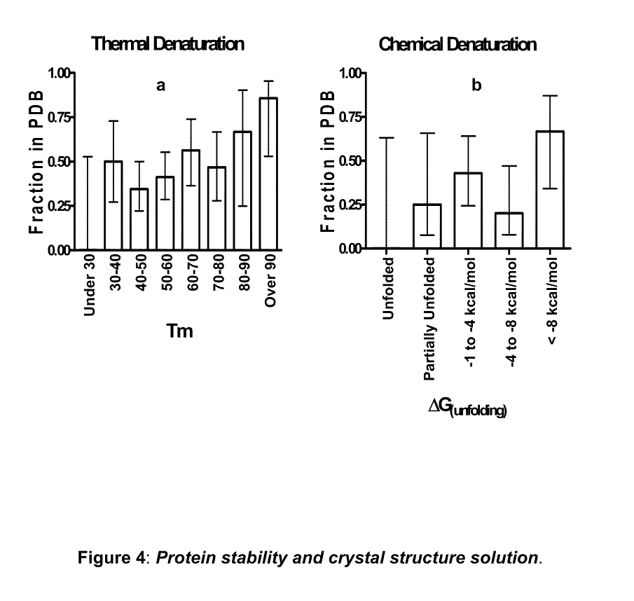

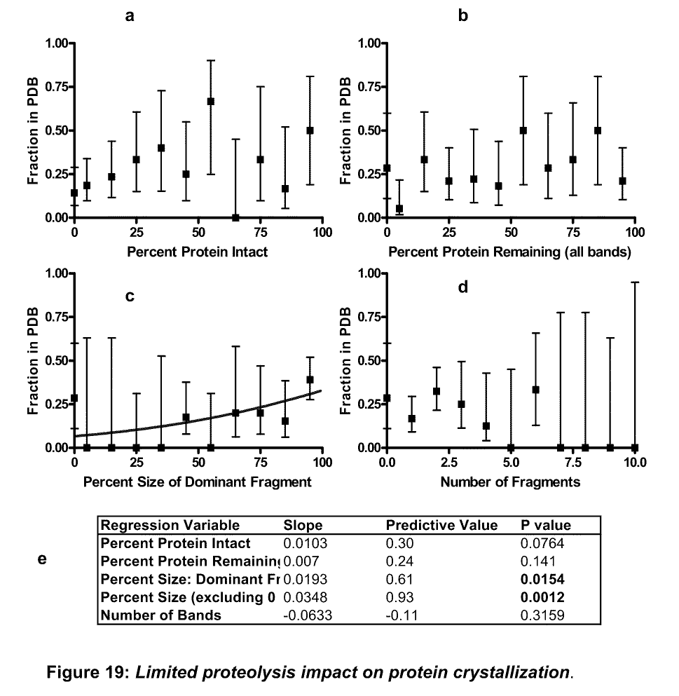

Several biophysical techniques were used to evaluate the relationship between protein stability and obtaining a crystal structure. 118 proteins were thermally denatured while observing the fluorescence of SYPRO Orange, a dye activated by contact with hydrophobic residues (FIG. 4a), 36 proteins were chemically denatured with guanidine hydrochloride while measuring circular dichroism (FIG. 4b), and 121 proteins were subjected to limited proteolysis and evaluated for instability as indicated by protease susceptibility (FIG. 19).

For thermal denaturation, protein stocks were diluted 1:40 in buffer containing 100 mM HEPES pH 7.5, 150 mM NaCl, and 5× SYPRO Orange (Invitrogen), to approximate final concentrations around 0.25 mg/ml. Proteins were heated in optically clear PCR tubes in a RT-PCR machine from 25 to 95° C. at 1 degree/min, with fluorescence measured each minute. SYPRO Orange fluorescence increases upon binding to hydrophobic residues, and sigmoidal cooperative denaturation is usually visible; Tₘ's were calculated as the inflection point of the fluorescence signal. CD spectra were taken for proteins with high initial signal or no visible denaturation. Proteins which showed no baseline before unfolding were considered partially unfolded in solution.

For chemical denaturation, proteins were diluted to 1 mg/ml, and CD spectra were taken from 190 to 300 nm to ensure native folding. Proteins were then titrated with a stock of 8M guanidine HCl and 1 mg/ml protein to a final concentration of 6M in an Aviv Model 202 machine, observing CD at 222 nm. Free energy of unfolding was calculated with Van't Hoff analyses. Proteins that showed no baseline before unfolding were considered partially unfolded in solution.

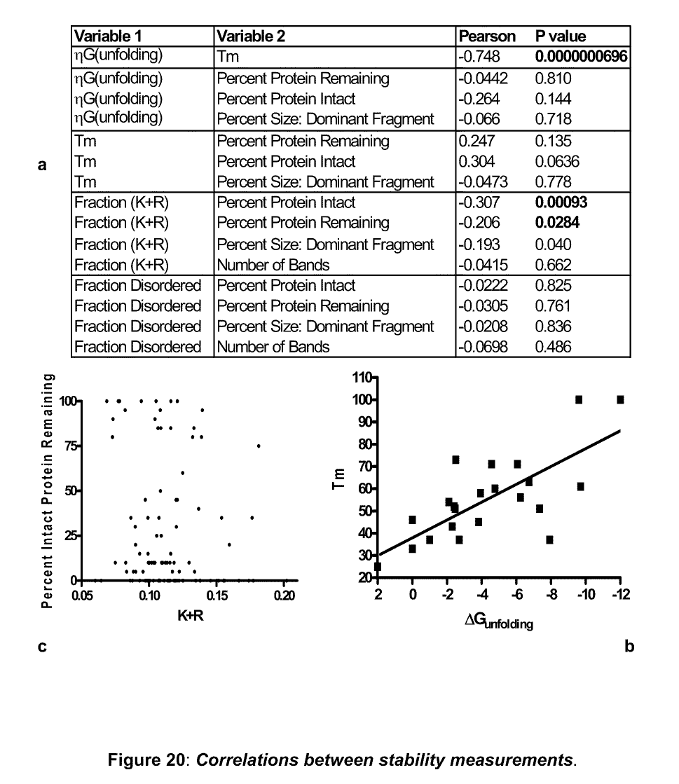

All three measures, which were significantly correlated (FIG. 20), indicated that very stable proteins were somewhat more likely solvable by x-ray crystallography, while proteins which were very unstable in solution gave structures at a lower, but not insignificant rate. Between these extremes, however, no significant effect of protein stability on the likelihood of crystal solution was observed.

### Example 2

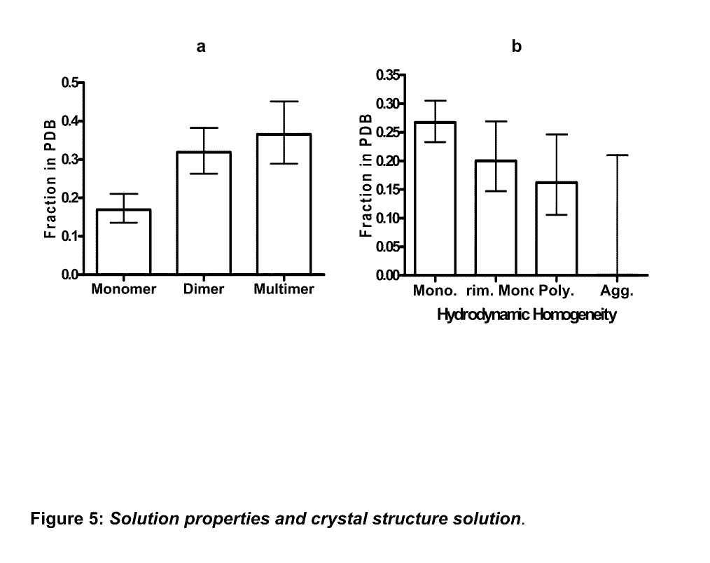

Correlation with successful structural determination was also determined for several biophysical characteristics other than stability, including oligomeric state, hydrodynamic homogeneity, and raw crystallization propensity. Monomers were shown to yield solvable structures less readily than dimers or multimers (FIG. 5a; significance in FIGS. 5a and 5b determined by evaluating contingency tables with a 2-tailed Fisher's exact test). This data, combined with earlier studies that synthetic oligomers can increase crystallization propensity, indicates that oligomeric symmetry may assist in crystal formation and subsequent structure determination. A protein's hydrodynamic homogeneity also influences crystal structure determination. Based on the protein set prior to culling proteins that were not predominantly monodisperse, proteins with 90% or more of a single species in solution were shown to be solved more than any others; proteins between 70% and 90% monodisperse were still significantly more likely to be solved than polydisperse proteins (FIG. 5b). Finally, the number of crystallization screen conditions which gave crystal hits was shown to be highly predictive of eventual structure determination (N=523, p_{logistic}=2.15×10⁻¹⁹). These observations are made after the protein has been expressed and purified, and not at the time of selecting or designing constructs. Nonetheless, these characteristics are shown to be correlated with successful structure determination, and their evaluation may aid in triaging proteins already in a crystallization effort, and may provide further understanding of the biophysical mechanics of crystallization.

### Example 3

Mutants of two proteins, PgR26 and RpR5, each with mutations at four different sites were engineered based on understanding of the crystallization mechanism metrics described herein. High-entropy hydrophobic amino acids in short surface-exposed loops were mutated to low entropy amino acids such as glycine and alanine. These engineered epitopes may aid protein crystallization by improving crystal packing contacts without sacrificing protein solubility.

For each protein, PgR26 and RpR5, secondary structure was predicted (e.g., using PhD/Prof). Sequence alignments of homologs were also generated (e.g., using PSI-BLAST server at NIH). The secondary structure and sequence alignments were aligned and sites in the combined alignment were identified that fulfilled predetermined criteria of (i) being in a particular secondary structure element (e.g., loops of 5 to 15 residue length), (ii) having a particular target residue (e.g., isoleucine, leucine, valine, glutamic acid, lysine), and (iii) having a particular replacement residue (e.g., glycine, alanine, phenylalanine). Amino acids at identified sites were mutated from target residue to replacement residue.

The proteins were expressed and screened for crystallization in parallel with wild-type protein. Resulting crystals were tested for diffraction resolution compared to crystals of wild-type protein.

For the PgR26 mutants, two new crystal forms were found. One mutant crystal form showed diffraction to 2.9 Å, compared to 8 Å for wild-type.

For the RpR5 mutants, several new crystallization conditions and one new crystal form were identified. The mutant crystal form showed diffraction to 2.8 Å, compared to 7 Å for wild-type.

Engineering results and the crystallization mechanism metrics described herein suggest that mutation of high SCE residues (including, but not limited to, isoleucine, leucine, valine, glutamic acid, and lysine) that are predicted to be surface exposed (for example, in short loops) to low entropy residues (including, but not limited to, glycine, alanine, and phenylalanine), in a way that preserves protein solubility, may positively affect crystallization—providing new and/or improved crystals. Proteins more amenable to crystallization may more readily yield atomic-resolution structures, which may be useful for further research and/or drug development.

### Example 4

This example provides a description of some embodiments of crystallization-improving mutagenesis (i.e., methods for producing engineered proteins for high-resolution x-ray crystallographic structure determination). The example provides the following steps (not necessarily performed in the order listed below):
1. Select a protein of interest.
   a. For example, one could select a protein which has crystallized, but done so poorly (i.e., the quality of the crystals is not suitable for high-resolution x-ray crystal structure determination). One could also select a protein which has not been possible to crystallize at all. Almost any protein can be selected (very short proteins, however, are unlikely to have suitable target amino acids due to their small size).
2. Perform secondary structure prediction on the selected protein of interest.
   a. In one aspect the PhD/PROF prediction suite could be used. This suite is available online as part of the PredictProtein server at predictprotein.org. Any other protein secondary structure prediction method/program could also be used for this purpose.
   b. If the secondary structure of the selected protein of interest is known, this step is optional.
3. Generate a sequence alignment of a homolog (or two or more homologs) of the selected protein of interest with the selected protein of interest.
   a. As an alternative, or in addition, one can align the selected protein of interest with one or more functionally identical proteins (orthologs). Thus, alignment of the selected protein of interest with one or more homologs and one or more orthologs can be used.
   b. Any routinely acceptable sequence alignment method could be used. For example, the PSI-BLAST server from the NIH would be useful for this step. In this instance, one can restrict the alignment to close homologs by only taking sequences with high alignment scores. Examples of other suitable programs include, but are not limited to BLASTp, available through the National Center for Biotechnology Information (www.ncbi.nlm.nih.gov), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14.
   c. If an alignment of the selected protein of interest with one more of its homologs and/or orthologs is known, this step is optional.
4. Based on the secondary structure prediction and/or a sequence alignment of the homologs (and/or orthologs) of the protein, look for positions which:
   a. Are predicted to be in the right secondary structure element (for example, look for loops (as opposed to helices or sheets) that are 6-15 amino acids long, which is where glycine's predictive signal is localized);
   b. Are occupied by the right type of target residue;
      i. for example, look to identify isoleucine, leucine, and valine, which are big hydrophobic residues;
      ii. as another example, look to identify glutamic acid and lysine if solubility is not a concern.
   c. Have a suitable replacement residue appearing in the sequence alignment of the protein of interest with one or more of its homologs and/or orthologs at that position:
      i. For example, the replacement residue can be glycine; but also could be alanine or phenylalanine;
      ii. Additional factors that can be taken into account are sequence conservation (e.g., if the wildtype residue is isoleucine, is it isoleucine most of the time in the alignment, or is it a fairly evolutionarily flexible residue) and prevalence of the desired amino acid (e.g., does glycine appear only once, or a few times).
      iii. For example, in order of desirability, these alignments would be increasing in order:
         1. IIIIIIIIIIIIIIIIIIIIIIII
         2. IIIIIIIIIIIIIIIVVVVVV
         3. IIIIIIIIIIIIIIIIIIIIIIIG
         4. IIIIIIVVVVVGG
         5. IAHYLGGEHA
         6. IGGGGGGGGGG
5. For larger proteins, typically there are a few suitable sites for replacement of a target amino acid with a replacement amino acid; one can choose one or more different sites for generation of the engineered protein.
   a. For example, one can make an engineered protein with single, double and/or multiple point mutations.
6. Generate the amino acid sequence for the engineered protein (i.e., protein of interest having one or more target amino acids replaced with one or more replacement amino acids).
7. Optionally, express the engineered protein (additional modifications to the engineered protein can be made, for example, to facilitate the expression or purification of the engineered protein). Standard protein expression and protein purification techniques known in the art can be used for this purpose.
8. Optionally, attempt to crystallize the expressed (and purified) engineered protein, and see if its crystallization properties improve relative to the selected protein of interest. Standard crystallization techniques known in the art can be used for this purpose.

### Example 5

### Exemplary Materials and Methods for Crystallization Engineering

Primary Datamining—For the 679 development proteins, protein sequences were culled from the SPINE database and manually curated to verify predominant monodispersity, based on Wyatt-AKTA static light scattering traces. For consistency, tags were removed from the protein sequences. The frequency of each amino acid, and the compound sequence metrics of charge, pI, GRAVY, SCE, length, and DISOPRED were individually regressed against the binary outcome of a PDB structure. Charge was calculated based solely on amino acid counts, considering only arginine, lysine, glutamic acid, and aspartic acid. Isoelectric point was calculated using the EMBOSS algorithm at ExPASy. GRAVY was calculated using the Kyte-Doolittle values of hydropathy. SCE values for the individual amino acids were taken from Creamer (2000). DISOPRED scores were calculated locally using the DISOPRED2 program with a 5% false positive rate. Calculations of predicted burial/exposure and secondary structure were performed with PhD/Prof. Exposed and buried fractions were calculated as fractions of total exposed or buried length; e.g., number of predicted buried alanines divided by the number of total predicted buried residues. For binned distribution graphs, bins were equally spaced and graphed by bin center; for terminal bins on unbounded variables, the bin center was calculated as the bin average.

Proteome Validations—Sequences with one or more predicted transmembrane helices, as predicted by the program tmmhmm were excluded from analysis. PDB sequences were downloaded and are from the SEQRES database of X-ray structures. For proteome validation logistic regressions, all predicted protein sequences were assigned a value of zero, to avoid problems with construct variation and single-domain structure determinations. Rolling Pₓ, averages were calculated using bins of 2000 proteins for both humans and E. coli.

Human Crystallization Metric (P_{xsH})—Proteome and PDB data were collected as above. Each sequence was randomly assigned to the Development set or the Validation set. Based on the enrichments of low-SCE residues shown in FIG. 17, multiple logistic regressions were run to analyze status as a PDB (1) or proteome (0) sequence based on fractions glycine, phenylalanine, and proline; fraction glycine among buried residues; fraction disordered residues; length; and the interaction terms of fraction disordered residues with the amino acid fractions. Insignificant variables were dropped from the regression and the regression was repeated to determine accurate coefficients. The combined metric was then used to predict and bin the Validation set.

Statistics—Logistic regressions were performed in S-PLUS, and significance was determined by 2-tailed T-test. The significance of oligomeric state, dispersity, and the dividing line in the charge/hydrophobicity chart (FIG. 12), was determined by evaluating contingency tables with a 2-tailed Fisher's exact test. Ninety-five percent confidence intervals were calculated using the binomial distribution.

Chemical Denaturation—Proteins were diluted to 1 mg/ml, and CD spectra were taken from 190 to 300 nm to insure native folding. Proteins were then titrated with a stock of 8M guanidine HCl and 1 mg/ml protein to a final concentration of 6M in an Aviv Model 202, observing CD at 222 nm. Free energy of unfolding was calculated with Van't Hoff analyses. Proteins which showed no baseline before unfolding were considered partially unfolded in solution.

Thermal Denaturation—Protein stocks were diluted 1:40 in buffer containing 100 mM HEPES, pH 7.5, 150 mM NaCl, and 5× SYPRO Orange (Invitrogen), to approximate final concentrations around 0.25 mg/ml. Proteins were heated in optically clear PCR tubes in an RT-PCR machine from 25 to 95 degrees at 1 degree/min, with fluorescence measured each minute. SYPRO Orange fluorescence increases upon binding to hydrophobic residues, and sigmoidal cooperative denaturation is usually visible; Tₘ's were calculated as the inflection point of the fluorescence signal. CD spectra were taken for proteins with high initial signal or no visible denaturation. Proteins which showed no baseline before unfolding were considered partially unfolded in solution.

What is claimed is:

1. A method of producing an engineered protein for high-resolution x-ray crystallographic structure determination, the method comprising:
(a) selecting a protein of interest;
(b) aligning the protein of interest with a homolog of the protein of interest;
(c) predicting the secondary structure of the protein of interest;
(d) identifying a target amino acid in the protein of interest that:
(i) is part of a secondary structure element in the predicted secondary structure of the protein of interest; and
(ii) is aligned with a replacement amino acid in the homolog of the protein of interest; and
(e) replacing the target amino acid in the protein of interest with the replacement amino acid to provide an engineered protein of interest.

2. The method of claim 1, further comprising expressing the engineered protein of interest in a cell.

3. The method of claim 2, further comprising crystallizing the expressed engineered protein of interest.

4. The method of claim 1, wherein the secondary structure element is a surface-exposed loop.

5. The method of claim 4, wherein the loop is 5 to 15 amino acids in length.

6. The method of claim 1, wherein the target amino acid is selected from the group consisting of: isoleucine, leucine, valine, glutamic acid, and lysine.

7. The method of claim 1, wherein the target amino acid is isoleucine, leucine, or valine.

8. The method of claim 1, wherein the replacement amino acid is selected from the group consisting of: glycine, alanine, and phenylalanine.

9. The method of claim 1, wherein the replacement amino acid is glycine.

10. The method of claim 1, wherein the replacement amino acid is phenylalanine.

11. The method of claim 1, further comprising generating a nucleic acid sequence encoding the engineered protein of interest.

12. A system for designing an engineered protein for high-resolution x-ray crystallographic structure determination, the system comprising a computer having a processor and computer-readable program code for performing the following method:
(a) selecting a protein of interest;
(b) aligning the protein of interest with a homolog of the protein of interest;
(c) predicting the secondary structure of the protein of interest;
(d) identifying a target amino acid in the protein of interest that:
(i) is part of a secondary structure element in the predicted secondary structure of the protein of interest; and
(ii) is aligned with a replacement amino acid in the homolog of the protein of interest; and
(e) replacing the target amino acid in the protein of interest with the replacement amino acid to provide an engineered protein of interest.

13. The system of claim 12, wherein the secondary structure element is a surface-exposed loop.

14. The system of claim 13, wherein the loop is 5 to 15 amino acids in length.

15. The system of claim 12, wherein the target amino acid is selected from the group consisting of: isoleucine, leucine, valine, glutamic acid, and lysine.

16. The system of claim 12, wherein the target amino acid is isoleucine, leucine, or valine.

17. The system of claim 12, wherein the replacement amino acid is selected from the group consisting of: glycine, alanine, and phenylalanine.

18. The system of claim 12, wherein the replacement amino acid is glycine.

19. The system of claim 12, wherein the replacement amino acid is phenylalanine.
